(19) United States
(12) Patent Application Publication
(10) Pub. No.: US 2011/0030693 A1
(43) Pub. Date: Feb. 10, 2011

(54) Oxygen mask

(76) Inventor: Po-Yu Kuo, Panchiao Taipei (TW)

Correspondence Address: PRO-TECHTOR INTERNATIONAL SERVICES, 20775 NORADA CT., SARATOGA, CA, 95070, US

(21) Appl. No.: 12/535,588

(22) Filed: Aug. 4, 2009

(51) Int. Cl. A61M 16/06; A62B 18/02

(52) U.S. Cl. 128/207.13

(57) ABSTRACT

The present invention discloses a structural improvement of an oxygen mask which covers a nose without covering a mouth, is small in size, has a lower cost and facilitates an oral treatment to a patient. In addition, a respiration tube of the oxygen mask faces upward to facilitate a physical examination to the patient.

### BACKGROUND OF THE INVENTION

a) Field of the Invention

The present invention relates to an oxygen mask and more particularly to an oxygen mask which covers a nose without covering a mouth, is small in size, has a better bonding ability and further facilitates an oral treatment and a physical examination.

b) Description of the Prior Art

Referring to FIG. 4, as covering a mouth A and a nose B of a patient simultaneously, a conventional mask 100 is provided with a larger area and an imperfect bonding ability; besides, a metal piece 101 must be used to clip a nose bridge. On the other hand, as the mouth A is covered by the mask 100, it is not convenient for treatment and as a respiration tube 102 faces downward, it is even inconvenient for the physical examination.

### SUMMARY OF THE INVENTION

Accordingly, the primary object of the present invention to provide an oxygen mask which covers a nose without covering a mouth, is small in size, has a better bonding ability, facilitates an oral treatment and is convenient for a physical examination as a respiration tube faces upward.

To enable a farther understanding of the said objectives and the technological methods of the invention herein, the brief description of the drawings below is followed by the detailed description of the preferred embodiments.

### BRIEF DESCRIPTION OF THE DRAWINGS

FIG. 1 shows a schematic view of the present invention.

FIG. 2 shows an exploded view of the present invention.

FIG. 3 shows a schematic view illustrating actions of the present invention.

FIG. 4 shows a schematic view illustrating actions of a prior art.

### DETAILED DESCRIPTION OF THE PREFERRED EMBODIMENTS

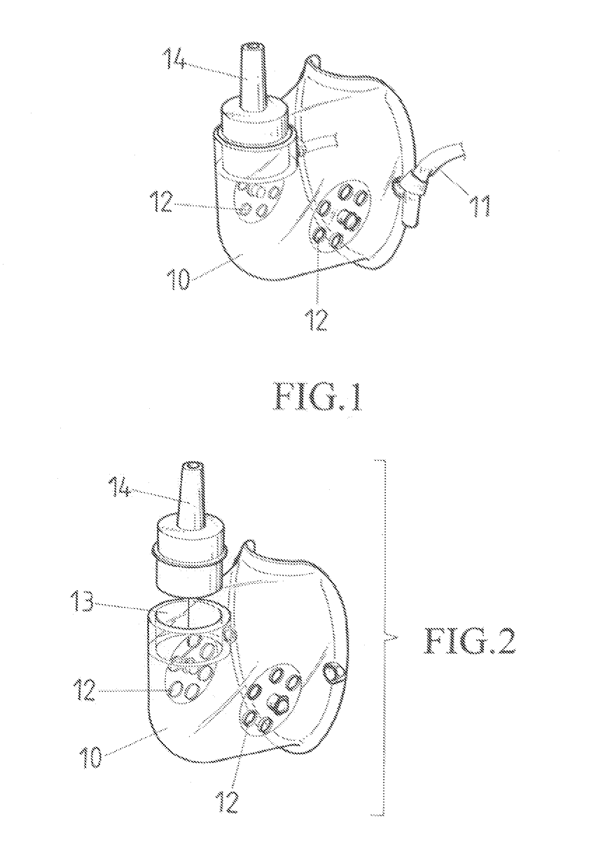

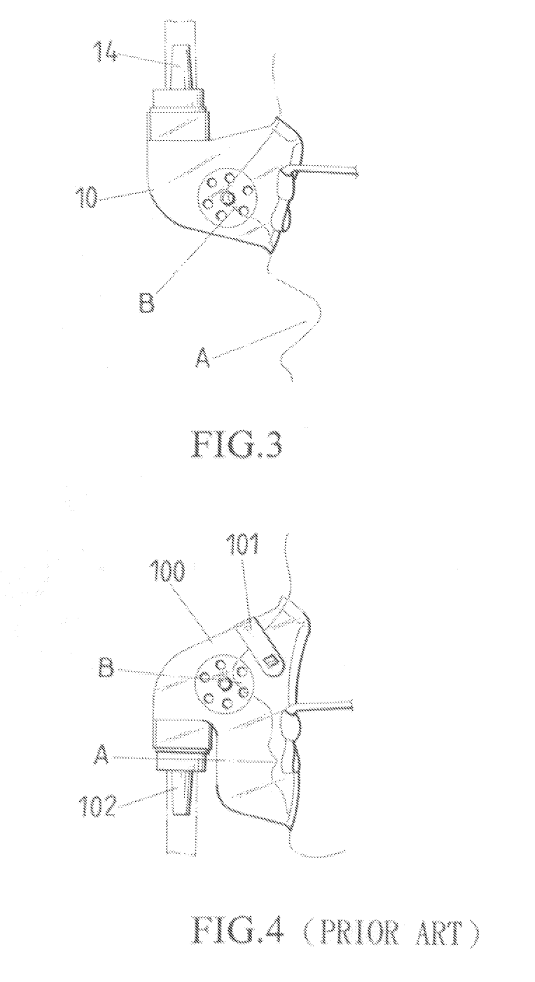

Referring to FIG. 1 and FIG. 2, an oxygen mask 10 of the present invention is small in size and only covers a nose B of a patient without covering a mouth A (as shown in FIG. 3); therefore, the oxygen mask 10 is provided with a good bonding ability and a lower cost, and facilitates an oral treatment. Openings at two sides of the mask 10 are tied with straps 11 to sheath the mask 10 at a patient's head, two ends of the mask 10 are provided respectively with an air vent 12, and a top part at a front side is provided with a latch slot 13 to latch a respiration tube 14. As the respiration tube 14 faces upward, a piping of the respiration tube 14 will not wind around a chest, thereby facilitating a physical examination.

Conclusive from the above, the present invention provides an oxygen mask which covers the nose without covering the mouth, is small in size, has the good bonding ability and facilitates the oral treatment. In addition to having the lower cost, the oxygen mask is convenient for the physical examination as the respiration tube faces upward

What is claim is:

1. An oxygen mask covering a nose without covering a mouth, a respiration tube being connected with the oxygen mask with a tube opening facing upward to facilitate a physical examination and a top end at a front side of the oxygen mask being provided with a latch slot to latch the respiration tube.

2. The oxygen mask according to claim 1, wherein a top part at a front side of the mask is provided with a latch slot to latch a respiration tube.

3. The oxygen mask according to claim 1, wherein two ends of the mask are provided respectively with an air vent.
